(19) United States
(12) Patent Application Publication
(10) Pub. No.: US 2025/0239368 A1
(43) Pub. Date: Jul. 24, 2025

(54) PREDICTING ONSET OF LABOR USING WEARABLE BIOMARKERS AND MACHINE LEARNING

(71) Applicant: ARIZONA BOARD OF REGENTS ON BEHALF OF THE UNIVERSITY OF ARIZONA, Tucson, AZ (US)

(72) Inventors: Elise ERICKSON, Tucson, AZ (US); Shravan ARAS, Tucson, AZ (US); Chinmai BASAVARAJ, Tucson, AZ (US)

(21) Appl. No.: 19/033,311

(22) Filed: Jan. 21, 2025

Related U.S. Application Data
(60) Provisional application No. 63/623,100, filed on Jan. 19, 2024.

(51) Int. Cl. G16H 50/20; G16H 50/70

(52) U.S. Cl. CPC G16H 50/20; G16H 50/70

(57) ABSTRACT

A platform that uses artificial intelligence and machine learning (AI/ML) to integrate wearable sensor data to detect shifts in the underlying biology that signals when the body is getting ready for labor and before symptoms (e.g., contractions) become prevalent. Shift detection may leverage a multimodal distribution of physiological markers. Physiological markers may include, for example, body temperature, heart/respiration or heart rate variability, sleep, circadian or ultradian patterns, caloric expenditure/activity, etc. A predicted time before delivery may give the user a due date that is based on real-time physiological data gleaned from a wearable device.

### CROSS-REFERENCE TO RELATED APPLICATIONS

This application claims priority to U.S. Prov. Pat. Appl. No. 63/623,100, filed Jan. 19, 2024, which is hereby incorporated by reference in its entirety.

### FEDERAL FUNDING

None

### INVENTIVE FIELD

The present invention relates to systems and computer-implemented methods for personalizing prenatal care and, more specifically, predicting the onset of labor using wearable biomarkers and machine learning (e.g., artificial intelligence).

### BACKGROUND

Despite advances in obstetrics, it is still not known exactly when a pregnant woman will begin labor. Due to their limitations, current clinical solutions still get the pregnant woman's due date wrong 95% of the time. The precise timing of the onset of labor remains a stubbornly unanswered question. As such, the inability to predict labor remains a barrier to safely addressing many adverse pregnancy outcomes. Current obstetric practice ascribes an “estimated delivery date” of 40.0 weeks to every pregnancy. That estimated delivery date, however, bears no individual-level predictive power as only 5 percent of labors occur on this date. As shown in FIG. 1A, most pregnancies will shift away from uterine quiescence into labor between 37-42 weeks gestation, giving rise to the 40-week averaged expected delivery date. Worldwide, adverse pregnancy outcomes rise considerably within the preterm and post term windows. However, adverse outcomes also occur within the span of “term,” linked to unanticipated pregnancy conditions (e.g., preeclampsia) or social determinants of health (e.g., access to care). Accordingly, addressing the uncertainty of the traditional, imprecise expected delivery data by determining a physiologically informed forecast of future labor onset would fundamentally transform obstetric research and care.

The pregnancy due date lacks precision, which intensifies uncertainty in providing obstetric care. In an era of personalized medicine, pregnant individuals deserve a more personalized due date. The estimated delivery date construct was originally based on a population average length of gestation; thus, providing no personal predictive value. The consequences of this imprecision are costly as giving birth preterm (<37 weeks) poses tremendous challenges to families and health systems. For those that reach term, anxiety about prolonged pregnancies (going “overdue”) leads to extra healthcare costs with higher labor induction and Cesarean rates. The uncertainty of when labor could begin can be complex and anxiety-provoking. In addition, a growing number of hospitals are ceasing to provide maternity care, therefore, all people living far from a facility have the added challenge and calculus of getting to a hospital on time. In fact, unexpected birth at rural emergency departments that lack trained obstetric providers is a key driver of health outcome disparities for rural residents as people end up in ill-prepared hospitals or deliver at home. Labor onset prediction would provide significant benefits to individuals and families and could lead to a fundamental shift in the way in which we approach birth care.

Maternal morbidity, preterm birth, and timely access to care could be addressed by reducing uncertainty in labor onset. The 2022 March of Dimes “Report Card” gave the United States a “D+” grade, with an overall 10.7 percent preterm birth rate, up from 9.8 percent in 2011, and widening racial disparities. In addition, the March of Dimes estimated that 28.5 percent of pregnancies had inadequate prenatal care, which could be important contributors to disparities. Between 2006 and 2020, over 400 maternity hospital units were closed across the nation, leaving families in “maternal care deserts”. In 2022, the March of Dimes indicated that 6.9 million women and nearly 500,000 births (approximately 1 in 8) are among those living in areas with low or no access to maternity care. Use of real-time or remote monitoring offers critical access to patients in their homes, helping to address gaps in care. Remote pregnancy monitoring for blood pressure and glucose have been studied, demonstrating improved outcomes over traditional in-person only systems.

The timing of normal term labor is determined, at least in part, by fetal readiness for labor and the maternal bodily response to the fetal signals. Through normal physiological processes, the placental and fetal brain/adrenal maturation prepares the lungs for extrauterine gas exchange and signals readiness for labor. Because of this process, the length of gestation varies from person-to-person. In a typical normal term process, hormones from maturing fetal adrenal glands lead to changes in placental hormone production, hormones from placenta and adrenals lead to changes in lung function to support breathing after birth, brain growth and development also stimulates adrenal gland hormone secretion, aiding in postnatal blood sugar and temperature regulation, and maternal tissues receive and respond to the signals, contributing to regular uterine contraction and cervical dilation leading to labor and birth. Current clinical solutions cause anxiety for families and care providers alike which leads to both overuse and underuse of obstetric care. For example, labor induction before the due date is used to bypass uncertainty, travel time is increased to access hospitals for rural residents, morbidity/mortality rates rise for mothers (post “term”) and newborns (preterm), and unplanned home births or other locations. The reality is that current methods for predicting labor lack precision (e.g., everyone's due date is 40 weeks), symptoms like contractions are non-specific, and there are the prior art offers no reliable tests for preterm or term labor/birth in advance of actual labor.

Significance of proposed research methodology is rooted in the complete inability to predict timing of labor in current obstetric practice. The 40-week estimated delivery date does not tell pregnant individuals when they will likely give birth. In fact, the perception of the normal length of human gestation has not been clinically updated or improved upon since the development of “Nagel's rule” in the early 1800s. The rule of 280 days from the last menstrual period can be adjusted with early ultrasound data. Like Nagel's rule, however, that adjusted date only provides a demarcation of the duration of pregnancy, not when it is likely to end for a given person. In a study of over 15,000 women reaching 41 weeks gestation, the available maternal data (age, weight etc.) was not much better than a flip of a coin in predicting when labor would start. Advanced cervical changes often predict success with labor induction but not when labor will occur spontaneously. Presently, women are told to report symptoms, which requires distinguishing vague symptoms of discomfort from true labor and yields high rates of false positives. Unfortunately, advanced symptoms of labor do not typically provide adequate time to intervene in preterm labor prevention.

### SUMMARY

The disclosed technology provides a forecast predicting when labor might start to help manage uncertainty. Additionally, predicting the onset of labor impacts decision-making around work or school, travel, and high-risk condition management among expectant mothers and their families. Embodiments of the disclosed aims to innovate the concept of the “due date” and improve obstetric care and life for expecting families. The disclosed embodiments aim to solve current problems, including uncertainty of labor onset affects every pregnant woman and family from worries over preterm birth to going “overdue”, preterm birth (e.g., being born <37 weeks, which results in massive costs and health consequences). pregnancies that are prolonged can have more complications, rural residents often need to travel to deliver, planning (e.g., work, travel, leave, childcare), and, for some high-risks births, whether women should avoid labor. The disclosed technology seeks to validate a deep learning model of maternal physiological changes preceding labor, to establish a foundation for methods to improve preterm birth prediction and term delivery planning.

The disclosed technology is directed to wearable devices and eHealth tools for predicting labor onset and personalizing prenatal care. In some embodiments, the disclosed technology provides a platform that uses artificial intelligence and machine learning (AI/ML) to integrate wearable sensor data to detect shifts in the underlying biology that signals when the body is getting ready for labor and before symptoms (e.g., contractions) become prevalent. Shift detection may leverage a multimodal distribution of physiological markers. Physiological markers may include, for example, body temperature, heart/respiration or heart rate variability, sleep, circadian or ultradian patterns, caloric expenditure/activity, etc. A predicted time before delivery may give the user a due date that is based on real-time physiological data gleaned from a wearable device.

In some embodiments of the disclosed technology, a timely forecast for future labor onset may be provided. A timely forecast for future labor onset may improve, inter alia, hospital staffing, decision-making about individual patients, hospital bed space, and opportunities to develop new preterm labor interventions or prevention. A benefit of the disclosed technology is that it takes the guesswork out of labor. With an accurate prediction of labor onset, patients get to the right place at the right time. Additional benefits include, for example, improved prediction of labor and health system resource use, personalization of care, personal delivery planning and rural access to care, and maximizing delivery and infant outcomes across the gestational age spectrum.

Non-invasive physiological data may be useful in discovering patterns associated with expected labor onset. For example, machine learning (ML) may be used to study and analyze associated patterns of physiological data (e.g., temperature, heart rate, sleep, activity, etc.) of pregnant women. In that embodiment, a training dataset may be created based on the group of pregnant women while wearing a device (e.g., a smart ring) during third trimester and collecting physiological data from the device. In that embodiment, the likelihood of labor starting before 40 weeks versus passing the due data using maternal physiological data gathered from the device worn can be determined. That exemplary implementation provides a moderate ability to discern pregnancy passing 40 weeks from those laboring before the due date. The machine learning model in this example may include a boosted random forest approach. Additionally, training and testing dataset may be created by gathering wearable device information during the span of time from enrollment until four days before labor started or 40 weeks, whichever occurred first.

### BRIEF DESCRIPTION OF THE DRAWINGS

Aspects of exemplary embodiments may be better understood with reference to the accompanying drawings. The components in the drawings are not necessarily to scale, emphasis instead being placed upon illustrating the principles of exemplary embodiments.

FIG. 1A is a graph illustrating the distribution of labor onset relative to the traditional, 40-week estimate delivery date.

FIG. 1B is a block diagram of an architecture of the disclosed systems according to exemplary embodiments.

FIG. 2 is a block diagram of an exemplary system for predicting the onset of labor using a biomarker according to exemplary embodiments.

FIG. 3 is a block diagram illustrating a convolutional autoencoder of the system of FIG. 2 according to exemplary embodiments.

FIG. 4 is a block diagram illustrating an LSTM network of the system of FIG. 2 according to exemplary embodiments.

FIG. 5 is a block diagram illustrating a segmentation/aggregation module of the system of FIG. 2 according to exemplary embodiments.

FIG. 6 is a block diagram illustrating a multivariate system according to exemplary embodiments.

FIGS. 7A-7D depict the average error of an example system.

FIG. 8 is a graph of prediction performance an example system with respect to average daily error rate and days until labor onset.

FIG. 9 is a graph of the predicted gestational ages, generated by an example system, at natural labor onset and following labor induction.

FIG. 10 is a diagram illustrating a prediction window generated by an example system.

### DETAILED DESCRIPTION

Reference to the drawings illustrating various views of exemplary embodiments is now made. In the drawings and the description of the drawings herein, certain terminology is used for convenience only and is not to be taken as limiting the embodiments of the present invention. Furthermore, in the drawings and the description below, like numerals indicate like elements throughout.

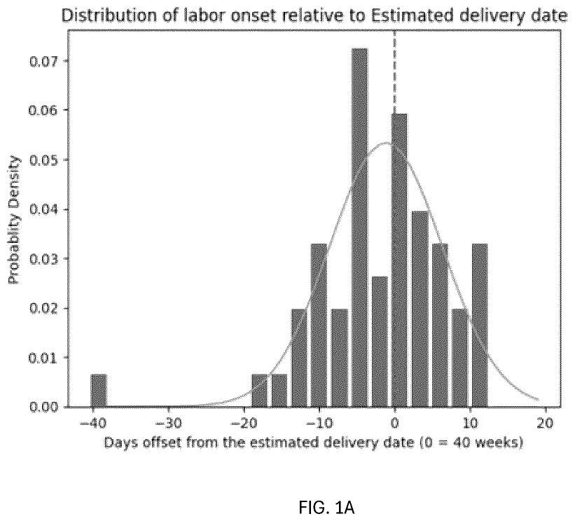

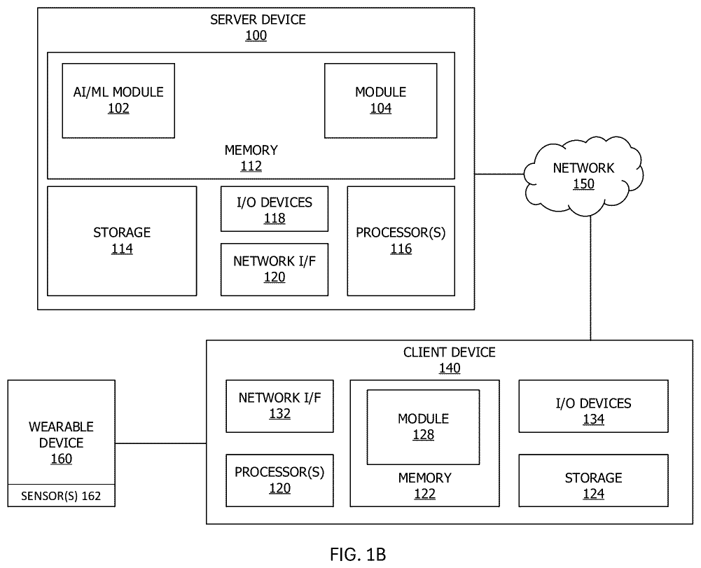

FIG. 1 is a block diagram of an architecture of the disclosed system according to exemplary embodiments.

As shown in FIG. 1, the architecture includes a client device 140 connected to one or more server devices 100 over a network 150. Additionally, or alternatively, client device 140 may be connected to a wearable device 160. While a single client device 140 is shown, the architecture may include multiple client devices 140 that are not shown for clarity. The client device 140 may be a personal computer, a tablet device, a smart phone, laptop computer, mobile device, network device, or any other electronic device which may be connected to, or paired with, wearable device 160. In some embodiments, the client device 140 and the wearable device 160 may be associated with a single user.

The network 150 may include one or more wired or wireless networks, wide area networks (e.g., the internet), local area networks, short range networks, etc. While shown as a single entity, the architecture may include multiple networks 150 and devices that are not shown for clarity. For example, the network 150 may include a wireless local area network accessible from a client device 140 via a wireless access point that is coupled, via a wired portion of the local area network to a router that is in turn coupled to the internet. The internet may include various sub-networks and protocols along with various versions of these sub-networks and protocols and various hardware components (such as servers, switches, routers, bridges, etc.) that provide the services for the internet. A user of the client device 140 can interact with the server device 100 to access services controlled and/or provided by the server device 100.

Client device 140 may include one or more processors 120. Examples of processors include a central processing unit, processor cores, image processors, microprocessors, graphic processing units, etc., which can execute computer code or computer instructions. The processor 120 may include multiple processors of the same or different type. For example, multiple processors 120 may be included as processor cores on a single processor package or chip. Multiple processor cores may also be integrated into system on a chip (SOC) packages, which often include various peripheral controllers, memories, interfaces, etc. on a single chip. The client device 140 may also include memory 122. Memory 122 may each include one or more different types of memory, which may be used for performing functions in conjunction with processor 120. For example, the memory 122 may include cache, ROM, RAM, or any kind of transitory or non-transitory computer readable storage medium capable of storing computer readable code. As used herein, non-transitory computer readable storage medium generally refers to computer accessible memory which can maintain data stored thereon for a period of time after power is removed. The Memory 122 may store various programming modules and applications 128 for execution by processor 120.

The client device 140 may also include a network interface 132 and input/output devices 134. The network interface 132 may be configured to allow data to be exchanged between the client device 140, the wearable device 160, the server device 100, and/or other devices coupled across the network 150. The network interface 132 may support communication via wired or wireless data networks. Input/output devices 134 may include one or more display devices, keyboards, keypads, touchpads, mice, scanning devices, voice or optical recognition devices, or any other devices suitable for entering or retrieving data by client device 140.

The server device 100 may include similar components and functionality as those described with reference to the client device 140. The server device 100 may include, for example, one or more servers, network storage devices, additional client devices, and the like. Specifically, the server device 100 may include memory 112, storage 114, one or more processors 116, one or more input/output devices 118, and a network interface 120. The Memory 112 may include one or more modules, such as software modules 104 and AI/ML modules 102.

The wearable device 160 may include similar components and functionality as those described with reference to the client device 140. The wearable device 160 may include, for example, any type of electronic device used to collect and transmit information about the wearer. For example, the wearable device 160 may be a smartwatch, smart glasses, jewelry (e.g., smart ring), etc. Information may be collected, for example, by one or more sensors 162. Information may include physiological data, activity tracker data, etc. Physiological data collected may include, for example, heart rate, blood glucose, blood pressure, respiration rate, body temperature, etc. In some embodiments, data collected by the one or more sensors may be collected and transmitted to the client device 140 and stored in the storage 124. Additionally, or alternatively, data collected by the one or more sensors 162 may be collected and transmitted to the server device 100 and stored in the storage 114. While the various components are presented in a particular configuration across the various systems, it should be understood that the various modules and components may be differently distributed across the network.

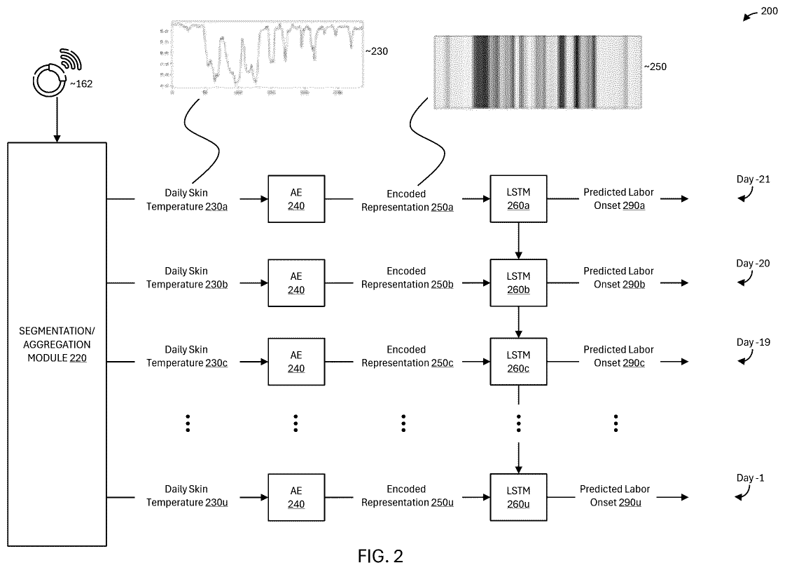

FIG. 2 is a block diagram of an exemplary system 200 for predicting the onset of labor using a personalized biomarker according to exemplary embodiments. In the example embodiment of FIG. 2, the system 200 predicts the onset of labor using daily skin temperatures 230.

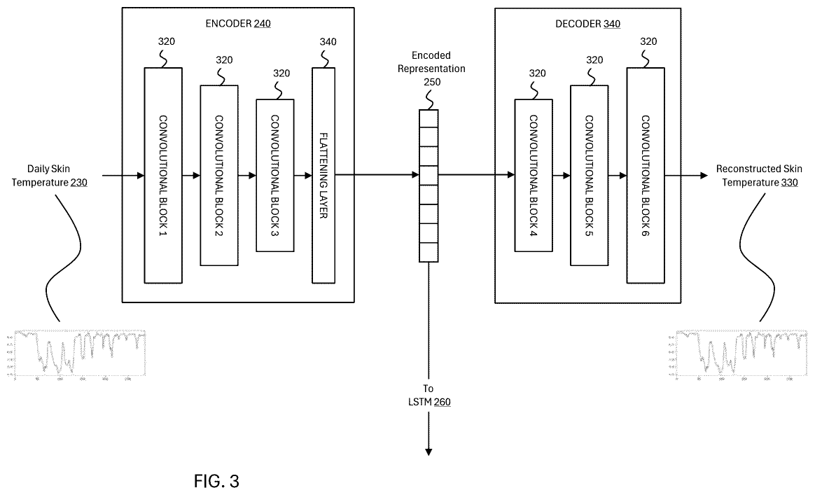

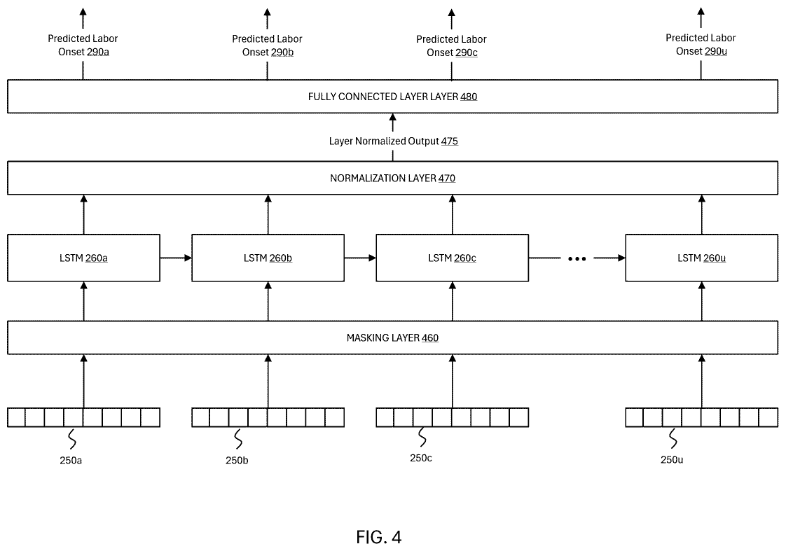

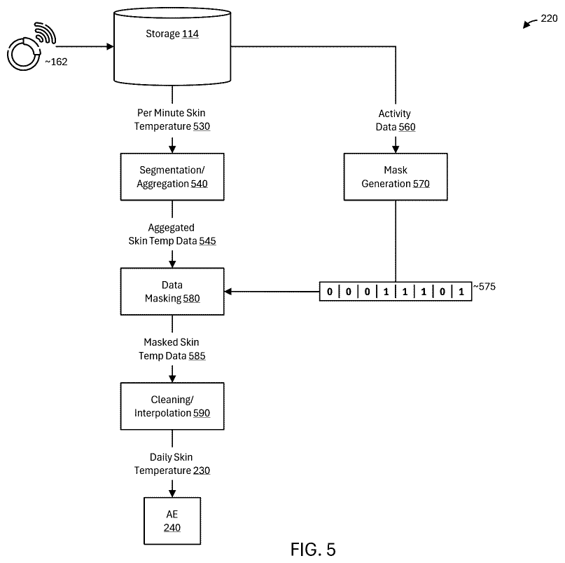

In the embodiment of FIG. 2, the system includes a segmentation/augmentation module 220 (described in detail, for example, with reference to FIG. 5) that provides daily temperature data 230 to convolutional auto-encoders 240 (described in detail, for example, with reference to FIG. 3), which extract encoded features and form encoded representations 250 of each daily temperature 230. Those encoded representations are feed into an AE-LSTM (Auto-Encoder Long-Short Term Memory) layer 260 (described in detail, for example, with reference to FIG. 4) to obtain a “days until labor onset” value 290 relative to the current gestational age. As shown in FIG. 2, for example, for each day leading up to labor, the system 200 may generate an encoded representation 250 of each daily temperature 230 (individually identified as encoded representations 250a, 250b, etc., of each as daily temperature 230a, 230b, etc.) and provide each of encoded representation 250 to an LSTM network 260 (individually identified as encoded LSTM cells 260a, 260b, etc.) that generates a predicted labor onset value 290 on each day leading up to labor (individually identified as predicted labor onset 290a, 290b, etc.)

Using high-frequency skin temperature data 230, the disclosed system 200 can predict an accurate future window for labor onset. Using a training dataset, the neural network may be trained and tested for predicting labor onset across gestation. For example, deep-learning methods may be used to analyze skin temperature 230 in Celsius recorded every minute from 54 participants who experienced a spontaneous labor. Participants' data may be segmented by day starting at day 240 of gestation and averaged skin temperature over 5-minute non-overlapping moving windows. The model uses skin temperature data 230 to recognize unique patterns for each day leading up to labor onset, regardless of their gestational age. The model may be fine-tuned using parameters (such as number of hidden layers, optimization algorithm, and regularization).

The disclosed system 200, for instance, may be presented with a time-series of daily (24 hours) skin temperature values 240 averaged across 5 minutes as input and outputs the time to labor onset 290 in days relative to current gestational age. First, the convolutional autoencoder 240 is used to generate a low-dimensional encoding 250 that efficiently captures temporal relations inherent in the daily skin temperature data starting from day 240 of gestation until the day before labor started (between 37-42 weeks). Each daily encoded representation 250 preceding labor onset is then fed to a long short-term memory (LSTM) network 260 that captures changes in skin temperature across days. The LSTM network 260 outputs the days until labor (with the L1 error as |true days to labor-predicted days to labor|). Across the sample, the system 200 predicts the days until labor onset with an average error (difference from the true labor). By 7 days prior to labor onset, labor was predicted by the disclosed system 200 with an average error of 1.92 days.

FIG. 3 is a block diagram illustrating the convolutional autoencoder 240 according to exemplary embodiments.

As shown in FIG. 3, the encoder 240 is paired with a decoder 240. The encoder 240 generates the encoded representation 250 of each daily skin temperature 230 by converting that data from a feature space to latent space, while the decoder 340 converts that encoded representation back to the feature space to generate a reconstructed skin temperature 330. Autoencoders train in an unsupervised setting, where the object loss function measures the loss of reconstructing the original signal from the latent representation.

In the exemplary encoder 240 of FIG. 3, for input data Tₖ of size 288 may be fed into a series of three convolutional blocks 320. Each convolutional block 320 may include a 1-D convolutional layer coupled with a max-pooling layer that enables reduction in data dimensionality. Output from the final convolutional layer 320 is flattened by a flattening layer 340 and fed into a dense fully connected layer to produce the encoded representation 250. The decoder 340 is a mirror image of the encoder, except that the max-pooling layer is replaced by an up-sampling layer that gradually increases the dimensionality back to the original feature space. In the example of FIG. 3, for instance, convolutional block 1 may include 64 filters of filter size 7 and a pooling size of 2, convolutional block 2 may include 32 filters of filter size 5 and a pooling size of 2, and convolutional block 3 may include 16 filters of filter size 3 and a pooling size of 2. Similarly, convolutional block 4 may include 16 filters of filter size 3 and an up-sampling of 2, convolutional block 5 may include 32 filters of filter size 5 and an up-sampling of 2, and convolutional block 5 may include 64 filters of filter size 7 and an up-sampling of 2.

FIG. 4 is a block diagram illustrating the LSTM network 260 according to exemplary embodiments.

In the embodiment of FIG. 4, LSTM model takes the sequence of encoded representations 250 (e.g., 64-dimensional encoded vectors that each represent a daily skin temperature 230) as input and output a predicted labor onset value 290 indicative days till labor relative to the current gestational age. A masking layer 460 may be used to exclude zero values during analysis and zero-padding may be used to conform the input sequences to a uniform length. The output of the masking layer 460 is fed into an LSTM layer 260 that is recurrent in nature. The LSTM layer 260 may have, for example 128 units 260a, 26ab, etc., and tanh may be used as the activation function. A normalization layer 470, reduces the dependency on batches, improves model performance, and is best suited for sequence-to-sequence models, may be used to normalize each output of the LSTM layer 260. Finally, the layer normalized output 475 is fed into a dense, fully connected layer 480 (e.g., with 128 units and linear activation function) that outputs the predicted labor onset 290 indicative of the days remaining to labor relative to current gestational age.

The core of the DNN model may be formalized as a non-linear approximator F: Yₖ=F(Tₖ,θ)+δ. Given the input sequence of daily temperatures, Tₖ={T^{k-1}, T^{k-2}, T^{k-3} . . . . T¹} starting from the kth day of gestation where T^{k} is a sequence of aggregate 5-minute temperatures for that day, the model represented by a set of trainable parameters θ predicts a value Yₖ that indicates number of days until labor relative to current gestational age k. δ represents the parameters of regularization methods employed to avoid model over-fitting. Model F over the space of all nonlinear models minimizes the Mean Absolute Error objective function Σ|yₖ− [symbol] |/N for all training subjects N, where [symbol] is the predicted days until labor and yₖ is true days until labor at a gestational age of k. As DNNs are highly effective universal non-linear approximators, the disclosed system 200 may use a combination of convolutional AE coupled with an LSTM network to train model parameters θ and δ.

FIG. 5 is a block diagram illustrating the segmentation/aggregation module 220 according to exemplary embodiments.

In the embodiment of FIG. 5, raw per minute temperatures 530 received from the wearable device 160 are averaged (e.g., over a 5-minute window) and segmented (e.g., into 24-hour periods starting at 10 am each day), which allows the neural network model to learn from daily patterns (both day and night variation). As shown in FIG. 5, for instance, a segmentation/aggregation module 540 may segment and aggregate the raw per minute temperatures 530 to form aggregated skin temperature data 545.

Next, activity data 560 received from the wearable device 160 may be used to identify and remove data collected when the device was not worn (e.g., using 5-minute activity labels indicating wear/non-wear provided by the device 160). As shown in FIG. 5, for instance, a mask generation process 570 may be performed to generate a mask 575 indicative of whether the device 160 was worn during each time period and a data masking process 580 may be performed to exclude the time periods when the device 160 was not worn (as indicated by the mask 575) to form masked skin temperature data 585.

Finally, a cleaning and interpolation process 590 may be performed to generate the daily skin temperature 230 that is provided to the convolutional autoencoder 240. For instance, a linear interpolation method may be used to account for missing and non-wear daily data.

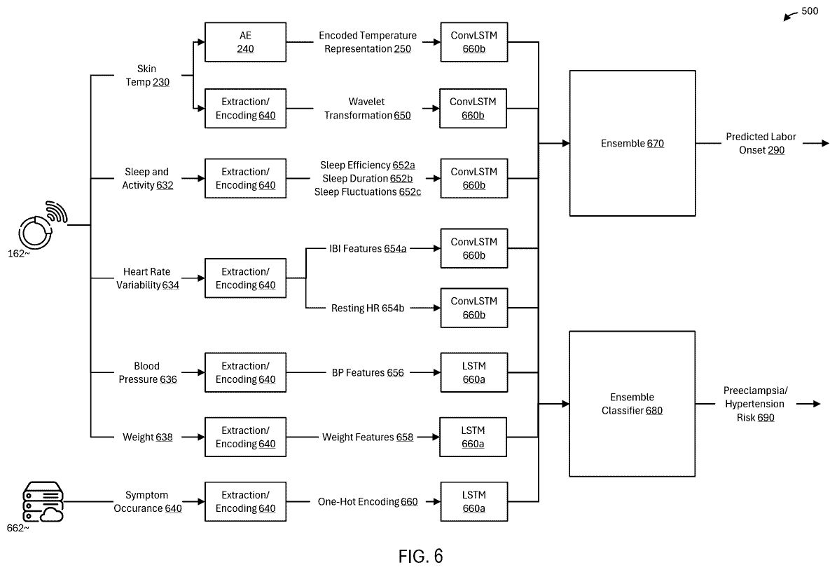

FIG. 6 is a block diagram illustrating a multivariate system 600 according to exemplary embodiments. As shown in the FIG. 6, features extracted from multiple biomarkers output by the wearable sensors 162 and/or symptoms 640 reported via an electronic health portal 662 may be used to generate the predicted labor onset 290 and/or a predicted risk 690 of preeclampsia or hypertension.

In the embodiment of FIG. 6, both time domain features (e.g., the encoded temperature representations 250) and frequency domain features (wavelet transformations 650) may be used for skin temperature 230. Feature extraction and encoding processes 640 may also be used to encode features extracted from sleep and activity data 632 (e.g., sleep efficiency features 652a, sleep duration features 652b, and/or sleep fluctuation features 652c), heart rate variability data 634 (e.g., resting heart rate 654b and/or time-domain and/or frequency-domain interbeat interval (IBI) features 654a such as root mean square of successive differences between normal heartbeats (RMSSD), standard deviation of normal (SDNN), number of normal sinus (NN) intervals that differ by more than 50 milliseconds from the previous interval, the percentage of successive normal cardiac interbeat intervals greater than 50 milliseconds (pNN50), the quantity and ratio of low-frequency (LF) and high-frequency (HF) components, etc.), blood pressure data 636 (blood pressure features 656), weight data 638 (weight features 658). Additionally, one-hot encodings 660 may be generated for each system occurrence 640 reported via an electronic health portal 662. Outputs from individual LSTM models 660a and/or convolutional LSTM models 660b may be pooled in an ensemble layer 670 that generates a predicted labor onset 290. Additionally, or alternatively, those features may be provided to an ensemble classifier 690 trained to classify individual as being at risk (or not at risk) of preeclampsia and/or hypertension. The ensemble layer 670 and/or the ensemble classifier 680 may use a bagging strategy (that uses majority voting between the individual models for each biomarker) a stacking strategy (where a complementary model is trained to learn how to best combine individual AE-LSTM predictions), or a boosting strategy (where the model prediction updated based on previous outcomes).

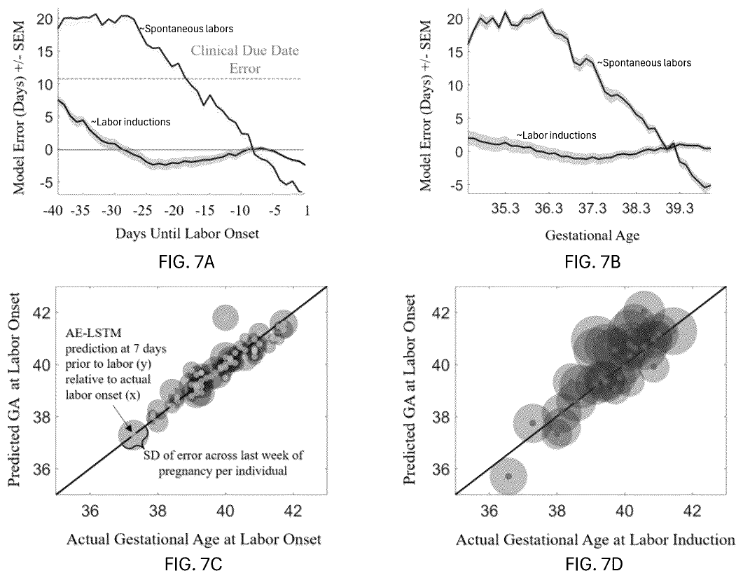

FIGS. 7A-7D depict the average error across the sample at each of 4 weeks prior to labor onset, with narrowing range of error as labor approaches. These data suggest that when a person is going through their pregnancy, temperature data could be used to predict a window for birth within a span of a few days that improves in accuracy over time. FIG. 7A shows modeling the error of spontaneous labors relative to errors in the model for induced labors. In FIG. 7A, the model error as measured starting at 40 days prior to labor onset or labor induction. In FIG. 4B, error estimates across range of gestation remains steady for pregnancies advancing toward spontaneous labor compared to the error among labor inductions, which is associated with advancing gestation. Errors for the induced participants was not expected to be predictive of labor onset given that the true future date of labor was unknown yet appear to track with gestational age (FIG. 7B). As seen in FIG. 7C, for spontaneous labor, particularly occurring prior to 41.5 weeks is predicted at 7 days prior to labor onset with very low error. In FIG. 7C, among spontaneous labors, the predicted gestational age at labor onset is shown with a solid circle (prediction made on-7 days) relative to actual labor date. Each shaded circle denotes the standard deviation (SD) of the error for each person across the last 7 days. FIG. 7D, in contrast, shows wide deviations in errors and a lack of precision in the estimate for future labor onset in induced participants. In FIG. 7D, among induced labors, prediction at −7 days from labor induction is shown in a solid circle and the standard deviation is shown in a shaded circle. Error is expected to be large as we do not know when labor would have begun had induction not occurred.

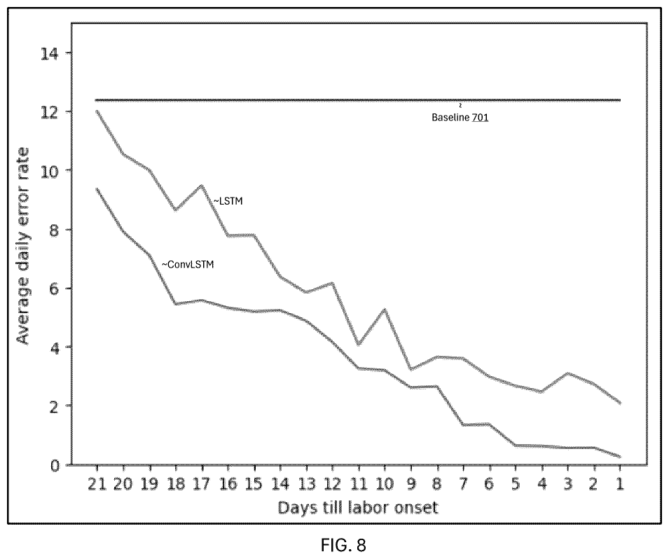

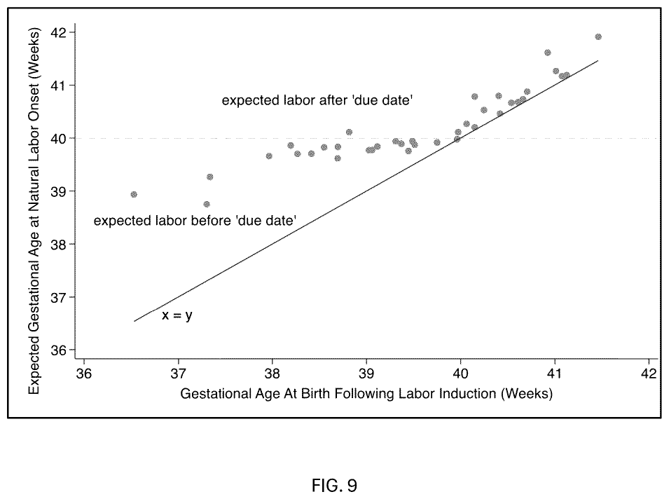

FIG. 8 is a graph of prediction performance an example system with respect to average daily error rate and days until labor onset. In this example, a convolution LSTM (ConvLSTM) AI model may be used to predict days until labor with a mean error of 3.6+/−1 days, outperforming a SimpleLSTM model that lacks convolutional embeddings. FIG. 9 illustrates a graph of the predicted gestational age at natural labor onset versus gestational age at birth following labor induction generated by an example system. FIG. 9 shows that the ConvLSTM AI model predicts labor onset (y-axis) vs. when they were induced (x-axis). For those before due date, the model predicts that induction was done at an early stage.

In some embodiments of the disclosed technology, a window prediction method may be provided. The window prediction method may include converting a prediction out from AE-LSTM to labor window. To improve clinical interpretation, we converted the predicted days until labor value y_{T} returned by AE-LSTM at time T into window [W₁, W₂] such that model prediction falls within it, W₁<γ_{T}<W₂, with a given probability P. This was achieved by converting the discrete prediction errors (MAE) for all participants during cross-validation across all folds at each time point T into a distribution E (T) by calculating a Kernel Density Estimate. We tested E (T) for normality using the Shapiro-Wilk test. With E (T) modeling, a normal distribution with mean Y′_{T}, the area under the KDE curve between bounds (ϵ₁, ϵ₂) then gave us the probability P of model error lying between them. We define this window as W(P)T and calculated the window bounds as W₁=Y′_{T}−ϵ₁, W₂=Y′_{T}+ϵ₂ for a given probability P∈{0.7, 0.8, 0.9, 0.95} and reported the window size (W|=|W₁|+|W₂| across Y′_{T} within which the prediction was likely to fall.

While the DNN model has been formulated to output a single “days until labor” value, the MAE (difference between true and predicted) error may be used to measure model accuracy that cannot be easily converted into clinical friendly measures for model positive and negative predictability. To aid clinical interpretation the concept of a prediction window W(P)_T may be derived from the error distribution of the model at time T with a given probability P. Table 2 gives an overview of model validation using a predictive labor window W(P)T with P E {0.7, 0.8, 0.9, 0.95} and T∈{7, 10}. The corresponding window sizes |W| for T=7 where {3.7, 4.6, 6.0, 7.1}; at T=10 where {6.2, 7.4, 9.2, 10.4} days respective for each value in P. Given the limited sample size of our cohort, we used all participants with a spontaneous birth for this analysis (N=54). A true positive (TP) may be defined as the number of participants (N) whose labor correctly started within the window predicted by the model. False positives (FP) may be defined as those participants whose labor occurred after the model prediction window. For these individuals, the model falsely predicts their labor to start in an earlier window as compared to true labor. It may be argued that this has a lesser impact on participant risk (they arrive earlier at the delivery center if following model prediction window) when compared to False Negatives (FN). False negatives are defined as those participants who go into labor before the predicted labor window by the AE-LSTM models. These participants would potentially be unprepared for labor if following model predictions. For both FP and FN, it is reported by how much the true labor was missed. This is calculated as the difference between true labor and edge of the prediction window (right edge for FP, left edge for TP). This is indicated by “False Positive Window Days” and “False Negative Window Days” respectively in Table 1. The model can predict with a 79% TP, 18.8% FP and a small 1.8% FN rate 10 days prior to actual labor when given a window size of 7.4 days. Naturally increasing the window size, also increases the TP, and reduces both FP and FN, however a larger window size (e.g., [W]=9.2, TP=96%) may not give pregnant mothers enough granularity to plan for labor. Similarly, it may be seen that a

TABLE 1

| | | | False Positive Window | | | False Negative Window | | |
|---|---|---|---|---|---|---|---|---|
| | | True Positive | Labor occurred after the | | | Labor occurred before | | |
| Prediction made | | Labor occurred | prediction window | | | the prediction window | | |
| Days to | Prediction | within the | | | False Positive | | | False Negative |
| Labor | Window | window. | | | Window Days | | | Window Days |
| Onset | \|W\| (days) | n (%) | n (%) | | mean ± SD | n (%) | | mean ± SD |
| −10 days | 6.2 | 36 (67) | 13 | (24.5) | 1.0 ± 0.6 | 4 | (7.5) | 0.6 ± 0.5 |
| | 7.4 | 42 (79) | 10 | (18.8) | 0.6 ± 0.5 | 1 | (1.8) | 0.9 ± 0.0 |
| | 9.2 | 51 (96) | 2 | (3.7) | 0.5 ± 0.4 | 0 | (0) | NA |
| | 10.4 | 52 (98) | 1 | (1.8) | 0.3 ± 0.0 | 0 | (0) | NA |
| −7 days | 3.7 | 40 (75) | 9 | (16.9) | 0.9 ± 0.6 | 4 | (7.5) | 0.8 ± 0.6 |
| | 4.6 | 42 (79) | 8 | (15.0) | 0.5 ± 0.5 | 3 | (5.6) | 0.7 ± 0.5 |
| | 6.0 | 49 (92) | 3 | (5.6) | 0.5 ± 0.4 | 1 | (1.8) | 0.5 ± 0.0 |
| | 7.1 | 51 (96) | 2 | (3.7) | 0.2 ± 0.1 | 0 | (0) | NA |

TP = 79%, FP = 15%, FN = 5.6% when using a 4.6-day predictive window, 7 days prior to true labor.

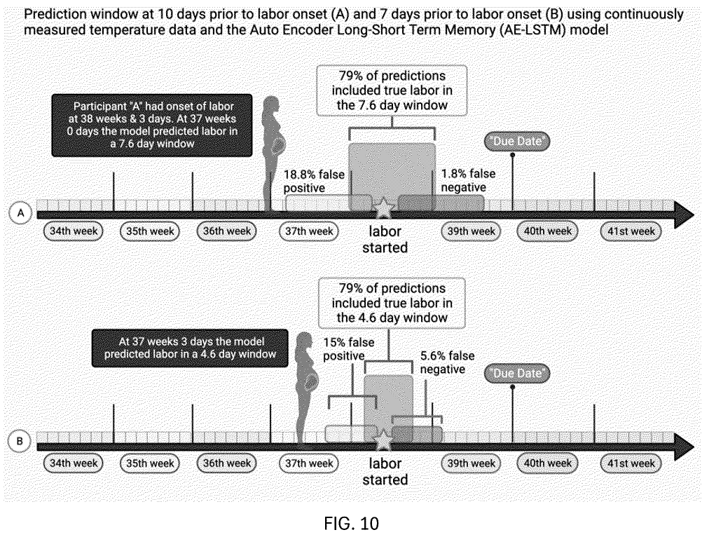

FIG. 10 illustrates a prediction window at 10 days prior to labor onset (A) and 7 days prior to labor onset (B) using continuously measured temperature data and the Auto Encoder Long-Short Term Memory (AE-LSTM) model.

### IMPLEMENTATION EXAMPLES

Deep-learning methods may be used to analyze skin temperature in Celsius recorded every minute from a number of participants (e.g., 55-60) who experienced a spontaneous labor onset to predict and evaluate the remaining time until labor relative to the current gestational age. Participants' data may be segmented by day for 21 days prior to labor onset and averaged skin temperature over 5-minute non-overlapping moving windows. A cross-validation method may be used to evaluate the model performance in predicting labor, where participants data may be split into training, validation, and testing. The model may use training data to recognize unique temperature patterns for each day leading up to labor onset, regardless of where the participants were in their gestation. Validation data may be used for fine-tuning model parameters such as—number of hidden layers, optimization algorithm, and regularization. First, a convolutional autoencoder may be used to generate a low-dimensional encoding that efficiently captures temporal relations inherent in the daily skin temperature data. Each daily encoded representation preceding labor onset may then be fed to a long short-term memory (LSTM) network that captures changes in skin temperature across days-specifically 21 days, until labor. The LSTM network outputs the days until labor onset. An L1 error may be calculated as |predicted days till labor-true days until labor|. The convolutional LSTM network may be used to predict the days until labor onset with an average daily error of 3.6±1 days in the 3rd week prior to labor onset. This is a significant improvement over the current standard EDD which has an average daily error rate of 12.3±1 days. The average daily error lowers substantially as labor onset is approached. That is, when a person is going through their pregnancy, temperature data may be used to predict a window of future labor within a span of a few days.

Example—consider a person at 38 weeks' gestation with the model providing an estimate of labor onset in days (t), e.g., t=3 days. Considering the accuracy of the prediction, the range is t−1.7 to t+1.7 or approximately 38 weeks 1 days to 38 weeks 4 days. The estimated due date given to all women is 40 weeks of gestation. The disclosed model, therefore, would have allowed this person to better prepare to give birth about 10 days earlier than she was expecting using the current clinical estimation.

### Training Dataset Examples

Datasets may be created using non-invasive smart rings for high-frequency sampling of physiological data during pregnancy (Biological Rhythms Before & After Your Birth (BioBAYB)). In some embodiments, an auto-encoder long-short term memory (AE-LSTM) model (a form of neural network) trained on pregnancies that ended spontaneously between 36 to 41.5 weeks of gestation may be created. An auto-encoded data representations of high frequency skin temperature recordings may be used to predict labor onset at 7 days with 1.9 days error. A central hypothesis is that model application in new pregnant participants' data may provide accurate real-time labor prediction across gestation which will inform future clinical trials.

### Model Evaluation

A subject-oriented approach may be used to evaluate the model, where a participant's data is wholly used either for training or testing. To evaluate the performance of all the models and methods, a k-fold cross validation method may be employed, where the participants' data is split into k folds. The model may be trained using k−1 folds of data and the model performance is tested on the held-out kth fold. This process is repeated k times until fold has served as the test dataset at least once. Subject-wise cross validation is a more effective way of evaluating model performance as it ensures that the model does not see any part of the test participants' physiological data during training, and it also improves generalization to new participants' data. With time-series data, the performance metrics are evaluated as a function of time. The absolute or squared difference of model prediction is computed to the ground truth at a specific point in time and report the mean error across time. During data pre-processing step, the performance is evaluated of several interpolation methods (such as linear, cubic-spline, and inverse distance weighted (IDW) interpolation) by computing the mean of the absolute difference between the original skin temperature with the interpolated value. The AE performance is evaluated by its ability to reconstruct the original signal from the low-dimensional encoded latent space representation. The mean squared error of the reconstructed signal is computed with the original signal. The AE-LSTM model performance is computed by evaluating the mean absolute error in predicting labor onset relative to current gestational age with the true labor onset. Valid prediction baselines are established based on the current standard using EDD and comparing the AE-LSTM performance to the baseline models. To better interpret the model's performance clinically, all model predictions are considered that go past the actual date of labor as negative. A report of the total number of negative predictions is calculated as part of the model card.

The disclosed technology brings a novel deep learning approach to address a long-standing key question in reproductive biology and maternity care. Informed by reproductive mammalian biology, the disclosed embodiments detect physiological changes that signal impending labor in real-time using non-invasive wearable devices. The disclosed will fundamentally update thinking about the estimated delivery date by providing necessary validation for development of a clinical tool for transforming obstetric practice.

While preferred embodiments have been described above, those skilled in the art who have reviewed the present disclosure will readily appreciate that other embodiments can be realized within the scope of the invention. Accordingly, the present invention should be construed as limited only by any appended claims.

What is claimed is:

1. A neural network-enabled system of predicting the onset of labor, comprising:
a server that receives information indicative of daily skin temperatures of a patient from a wearable health monitor;
one or more convolutional autoencoders that encode the information indicative of the daily skin temperatures to form encoded representations of the daily skin temperatures of the patient; and
a long short-term memory (LSTM) network, trained on a dataset of encoded representations of the daily skin temperatures of past patients having past labors onset at past gestational ages, that predicts a gestational age at labor onset of the patient based on the encoded representations received from the one or more convolutional autoencoders.

2. A neural network-enabled system of predicting the onset of labor, comprising:
a server that receives information indicative of one or more physiological biomarkers of a patient; and
a neural network, trained on a dataset of physiological biomarkers of past patients having past labors onset at past gestational ages, that predicts a gestational age at labor onset of the patient based on the one or more physiological biomarkers of a patient.

3. The system of claim 2, wherein the information indicative of at least one physiological biomarker is received from a wearable health monitor.

4. The system of claim 3, wherein the physiological biomarkers include daily skin temperatures.

5. The system of claim 4, wherein the physiological biomarkers further include heart rate, respiration rate, heart rate variability, sleep quality, circadian patterns, ultradian patterns, caloric expenditure, and/or physical activity.

6. The system of claim 3, wherein the one or more physiological biomarkers further include symptom events reported by the patient.

7. The system of claim 2, wherein:
the system further comprises one or more autoencoders that encode the information indicative of the one or more physiological biomarkers of the patient to form encoded representations of the one or more physiological biomarkers; and
the neural network is trained on encoded representations of the physiological biomarkers of the past patients and predicts the gestational age at labor onset of the patient based on the encoded representations of the one or more physiological biomarkers of the patient.

8. The system of claim 2, wherein the neural network is a recurrent neural network.

9. The system of claim 8, wherein the recurrent neural network is a long short-term memory (LSTM) network.

10. The system of claim 9, wherein the LSTM network is a convolutional LSTM network.

11. A computer-implemented method of predicting the onset of labor, the method comprising:
receiving information indicative of one or more physiological biomarkers from a patient;
providing data indicative of the one or more physiological biomarkers to a neural network trained on a dataset of physiological biomarkers of past patients having past labors onset at past gestational ages;
predicting, by the neural network, a gestational age at labor onset of the patient; and
outputting the predicted gestational age at labor onset of the patient.

12. The method of claim 11, wherein the information indicative of at least one physiological biomarker is received from a wearable health monitor.

13. The method of claim 12, wherein the physiological biomarkers include daily skin temperatures.

14. The method of claim 13, wherein the physiological biomarkers further include heart rate, respiration rate, heart rate variability, sleep quality, circadian patterns, ultradian patterns, caloric expenditure, and/or physical activity.

15. The method of claim 12, wherein the one or more physiological biomarkers further include symptom events reported by the patient.

16. The method of claim 11, wherein:
the method further comprises encoding the information indicative of one or more physiological biomarkers, by a convolutional autoencoder, to form encoded data indicative of the one or more physiological biomarkers; and
providing the data to the neural network comprises providing the encoded data encoded by the convolutional autoencoder.

17. The method of claim 11, wherein the neural network is a recurrent neural network.

18. The method of claim 17, wherein the recurrent neural network is a long short-term memory (LSTM) network.

19. The method of claim 18, wherein the LSTM network is a convolutional LSTM network.
